(19) United States
(12) Patent Application Publication
(10) Pub. No.: US 2011/0020947 A1
(43) Pub. Date: Jan. 27, 2011

(54) CHEMICAL COMPONENT AND PROCESSING DEVICE ASSEMBLY

(75) Inventors: William Bedingham, Woodbury, MN (US); Ranjani V. Parthasarathy, Woodbury, MN (US); Michael E. Danielson, St. Paul, MN (US); John C. Faletti, Cottage Grove, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 12/595,534

(22) PCT Filed: Apr. 25, 2008
(86) PCT No.: PCT/US08/61490

§ 371 Date: Oct. 12, 2009

Related U.S. Application Data
(60) Provisional application No. 60/913,814, filed on Apr. 25, 2007.
(60) Provisional application No. 60/985,827, filed on Nov. 6, 2007.

(51) Int. Cl. G01N 1/00; G01N 33/00

(52) U.S. Cl. 436/174; 422/63; 422/64

(57) ABSTRACT

A substantially dimensionally-stable chemical component is assembled with a sample processing device via a computer-controlled apparatus. In one embodiment, surface mount technology is used to assemble the chemical component with the processing device. The chemical component may include, for example, chemicals used for sample preparation or detection, such as a reagent. In different embodiments, the chemical component may be a tablet, microtablet, lyophilized pellet, bead, film, and so forth. In some embodiments, the chemical components are stored within a carrier that packages a plurality of chemical components. The carrier may define a plurality of pockets, where each pocket defines a discrete space for holding at least one chemical component. In some embodiments, a robotic arm aligns with a pocket in order to locate and remove a chemical component from the carrier and transfer the chemical component to a chamber of a processing device.

### CROSS REFERENCE TO RELATED APPLICATIONS

This application claims priority from U.S. Provisional Application Ser. No. 60/985,827, filed Nov. 6, 2007, and from U.S. Provisional Application Ser. No. 60/913,814, filed Apr. 25, 2007, the disclosures of which are incorporated by reference in their entirety herein.

### TECHNICAL FIELD

The invention relates to a processing device, and, more particularly, a processing device including at least one chemical component.

### BACKGROUND

Some processing techniques include biological and/or chemical reactions that are sensitive to temperature variations. In these processing techniques one or more samples of material can be processed in a processing device with multiple chambers. Different portions of one sample, or different samples, can be processed substantially simultaneously within the multiple chambers. Although it may be possible to process samples individually in this manner and obtain accurate sample-to-sample results, individual processing can be time-consuming and expensive.

Certain reagents used in these processing techniques may be expensive and subject to degradation during preparation, storage, and/or use of the processing device. For example, biological reagents, such as enzymes, are stored in a glycerol solution at −20° C. or in a powder or freeze-dried form to increase storage stability. However, the powder or freeze-dried forms of such materials may be difficult to measure, and freeze-dried structures such as spheres are fragile and tend to disintegrate when handled by a user or manipulated by the processing device.

### SUMMARY

In general, the invention is directed to methods and systems for placing one or more chemical components, such as tablets, microtablets, lyophilized pellets, beads, and the like, within a processing device. In some embodiments, the methods and systems described herein are useful for assembling a substantially self-contained processing device, such as a microfluidic device, that contains at least one of the chemicals involved in sample preparation and/or detection. The processing device may be configured to receive a sample and conduct a particular procedure, such as the preparation of a biological sample or detection of a bacteria, microorganism or nucleic acid within the sample. In some embodiments, the chemical component includes a reagent. In other embodiments, the chemical component is any component that includes a chemical useful for at least one stage of the sample preparation or detection, such as a wash chemical.

The chemical components that are placed in accordance with the systems and methods described herein are substantially dimensionally-stable, and are, therefore, substantially mechanically stable compared to a chemical in a liquid form. For example, the chemical component may be substantially solid or a gel. In some embodiments, multiple substantially dimensionally-stable chemical components are integrated into an automated process for assembling chemical components into a processing device. The placement device used to assemble the chemical components into a processing device may include a robotic arm controlled by a computing device, and may be, for example, a surface mounting technology (SMT) device that is typically used in the electronics industry.

As described herein, under the control of a computing device, a robotic arm automatically retrieves a chemical component, e.g., from a carrier including a plurality of chemical components, and places the chemical component within a process chamber of a processing device. In some embodiments, the robotic arm includes a vacuum tip that couples to the chemical component via a suction force. The computing device may control the robotic arm via any suitable technique, such as a system that identifies the relative location of each process chamber into which a chemical component is placed via coordinates or fiducial markers.

In some embodiments, the chemical components are packaged in a carrier that includes multiple chemical components separated into discrete pockets. The chemical components may be manufactured and packaged in the carrier and subsequently incorporated into a processing device. This separation of the chemical component preparation and assembly into a processing device permits the chemical component manufacturing and assembly to be performed at separate sites, if desired.

In one embodiment, the invention is directed to a method comprising introducing a substantially dimensionally-stable chemical component into a chamber of a sample processing device, and at least partially sealing the chamber of the processing device.

In another embodiment, the invention is directed to a method comprising placing a substantially dimensionally-stable chemical component comprising a reagent in a chamber of a processing device via surface mount technology, and at least partially sealing the chamber of the processing device.

In another embodiment, the invention is directed to a method comprising forming a plurality of substantially dimensionally-stable chemical components, the chemical components comprising at least one sample preparation or detection chemical for a sample processing device, and packaging the plurality of chemical components in a carrier. The carrier defines a plurality of pockets for receiving at least one chemical component.

In another embodiment, the invention is directed to an assembly comprising a carrier, a plurality of substantially dimensionally-stable chemical components disposed within the carrier, a sample processing device, a robotic arm, and a controller to control the robotic arm to transfer at least one of the plurality of chemical components from the carrier to the sample processing device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic top view of a processing device.

FIG. 2 is a schematic diagram of a placement device retrieving a chemical component from a carrier including a plurality of chemical components.

FIG. 3 is a schematic diagram of a carrier including a plurality of chemical components, where the carrier is wound on a reel.

FIG. 4A is a schematic diagram of a placement device placing a chemical component into a process chamber of a processing device.

FIG. 4B is a schematic illustration of a chemical component within a process chamber of the processing device of FIG. 1.

FIG. 4C is schematic illustration of a process chamber that includes support members for retaining a chemical component within the process chamber.

FIG. 5 is a partial cross-sectional view of the processing device of FIG. 1 and illustrates a process chamber including a chemical component.

FIG. 6 is a flow chart illustrating an embodiment of a technique for placing a chemical component within a sample processing device.

FIG. 7 is schematic illustration of a processing device including a plurality of sample input chambers and a plurality of processing chambers.

FIG. 8 is a schematic illustration of a processing device including a plurality of sequentially arranged process chambers.

### DETAILED DESCRIPTION

A chemical component may be placed within a processing device to prepare, detect or analyze a sample in a procedure conducted by a processing device. Example procedures include preparation of a biological sample for, for example, DNA sequencing, and/or detection, diagnostic or analytical procedures, chemical, biological or biochemical reactions, and the like. Examples of such reactions include detection via thermal processing techniques, such as, but not limited to, enzyme kinetic studies, homogeneous ligand binding assays, and more complex biochemical or other processes that require precise thermal control and/or rapid thermal variations.

In accordance with the methods and systems described herein, a chemical component includes at least one chemical, such as a reagent or biological controls, utilized in at least one step of the sample preparation and/or detection technique (generally referred to herein as “sample manipulation”). Sample manipulation may include, for example, capturing a biological material containing a nucleic acid; washing a biological material containing a nucleic acid; lysing a biological material containing a nucleic acid, for example, cells or viruses; digesting cellular debris; isolating, capturing, or separating at least one polynucleotide or nucleic acid from a biological sample; and/or eluting a nucleic acid.

Examples of sample preparation techniques include nucleic acid manipulation techniques, such as, but not limited to, polymerase chain reaction (PCR); target polynucleotide amplification methods such as self-sustained sequence replication (3SR) and strand-displacement amplification (SDA); methods based on amplification of a signal attached to the target polynucleotide, such as “branched chain” DNA amplification; methods based on amplification of probe DNA, such as ligase chain reaction (LCR) and QB replicase amplification (QBR); transcription-based methods, such as ligation activated transcription (LAT), nucleic acid sequence-based amplification (NASBA), amplification under the trade name INVADER, and transcriptionally mediated amplification (TMA); and various other amplification methods, such as repair chain reaction (RCR) and cycling probe reaction (CPR). Nucleic acid amplification may include, for example, producing a complementary polynucleotide of a polynucleotide or a portion of a nucleic acid in sufficient numbers for detection. Detection includes, for example, making an observation, such as detecting a fluorescence, which indicates the presence and/or amount of a polynucleotide or nucleic acid.

The processing device is a device that includes a sample loading chamber and at least one process chamber including at least one preloaded chemical component that includes the chemicals used in at least one sample manipulation step, such as by a processing device in sample preparation and/or detection. Thus, the processing device is configured to receive a sample and perform one or more sample manipulation steps without the need for a user to introduce the chemicals for the particular sample manipulation step. In some embodiments, the process chamber is sized to process discrete microfluidic volumes of fluids, e.g., volumes of 1 milliliter or less, 100 microliters or less, or even 10 microliters or less. In those embodiments, the processing device may be referred to as a “microfluidic” processing device. In embodiments in which the processing device includes all the chemicals necessary to perform a particular reaction, the processing device may be referred to as a “substantially self-contained” processing device.

In one embodiment, the processing device includes a chemical component that includes chemicals to prepare a sample for detection of a target nucleic acid or microorganism, such as a bacterium (e.g., methicillin-resistant staphylococcus aureus), and/or detect the target microorganism or nucleic acid within the sample. The sample may be taken from a human or nonhuman patient, and a living or nonliving source. The detection may be made with the aid of a detection system that detects the results of processing a sample within one or more process chambers of the processing device. For example, the detection system may actively interrogate a process chamber of the device to detect fluorescent reaction products in the chambers as the device rotates. The detection may be qualitative or quantitative. Other detection systems may be provided to monitor, e.g., the temperatures or other properties of the materials in the process chambers of the processing device. In some cases, a DNA target is detected, where the DNA target may be from cells from a human patient, a nonhuman animal, plant or another organism and used to identify specific DNA sequences in the tested subject genome.

The chemical component may include a reactive or non-reactive reagent for a particular sample manipulation procedure, and may optionally include a matrix material, which may be soluble or insoluble in a particular sample manipulation procedure. The distribution of the reagent within the matrix may be substantially uniform or non-uniform. In one embodiment, the reagent is a biological reagent, such as, but not limited to an enzyme, primer or probe, which are often used in, for example, nucleic acid amplification and detection. In other embodiments, the chemical component may include other types of reagents, such as primers, probes or microspheres capable of binding a nucleic acid. Exemplary matrix materials include a water-soluble polymer, a carbohydrate or a combination thereof.

In some cases, the chemical component may include one “dose” of a reagent required for an assay or another biological, chemical, biochemical or other type of reaction. In other cases, the chemical component may include multiple doses of the reagent, such that the chemical component may be used for more than one reaction or procedure. In other embodiments, the chemical component may include one or more doses of chemicals for a wash solution, e.g., to wash proteins off of microbeads, where the beads captured the proteins from a sample solution. In some embodiments, the chemical component may include more than one type of chemical, e.g., in distinct layers or sections, such that different chemical actions may occur as the chemical component dissolves in the presence of a fluid. In some cases, a coating may be applied to the different chemicals in the chemical component in order to help time the dissolution process, e.g., different coatings may help reduce the dissolution rate, while other coatings may increase the dissolution rate of the chemical component. If the chemical component includes more than one type of chemical or more than one type of chemical component is disposed within a process chamber, the reaction of one type of chemical or chemical component may provide the conditions appropriate for the dissolution and reaction of another chemical. For example, dissolution of a first chemical component may provide an acid change that encourages the dissolution and reaction of another adjacent or downstream chemical component.

The chemical component may be at least partially dissolved within a process chamber, e.g., via a fluid present within a sample or another fluid introduced into the processing device. However, prior to introduction of the chemical component into the processing device, the chemical component is dimensionally-stable. In this application, a dimensionally-stable component is sufficiently robust and self-supporting to allow a person or an automated apparatus to manipulate the chemical component without damaging the chemical component to the extent that it is unusable for its intended purpose in the sample manipulation procedure. Thus, the chemical component comprises one or more doses of a chemical for a particular reaction in a substantially self-supporting form. For example, in one aspect, a dimensionally-stable chemical component is sufficiently mechanically stable, thereby permitting handling of the chemical component by a robotic arm or another computer-controlled apparatus without separating the chemical component into multiple parts. In some embodiments, the chemical component substantially maintains its shape and dimensions within about 5%, and in some embodiments about 1%, during handling and introduction of the chemical component into the processing device.

In one embodiment, the chemical component is substantially solid or a gel. For example, a chemical component may be dimensionally-stable gel, which may include up to 90% liquid in composition, more typically about 10% to about 60% liquid, and thus exhibit densities similar to liquids, yet have the structural coherence of a solid. A dimensionally-stable gel may be selected to include a percentage of a liquid that permits handling by a robotic arm without substantial degradation of the structure of the chemical component (e.g., the chemical component remains a single structure during handling by the robotic arm, as opposed to breaking into multiple portions). In addition, in some cases, an assembly site may have a relatively cool operating temperature, e.g., below room temperature, in order to facilitate handling of the chemical component (e.g., a gel chemical component).

In one particular embodiment, the substantially solid chemical component is defined by compacting particles, e.g., a powder form of a reagent. That is, pressure (e.g., about 15 megapascals (MPa) to about 200 MPa) may be applied to the particles to define a substantially dimensionally-stable and substantially solid component from the loose powder or particles. Examples of compacted chemical components include tablets or microtablets (e.g., tablets having a greatest dimension less than about 5 millimeters (mm), and more typically about 0.5 mm to about 3 mm). For example, a reagent in a powder form may be compacted to define tablets or microtablets via a tablet press.

In other embodiments the chemical component may be in the form of a film that includes a reagent dispersed within or applied thereon. The film may be rigid or flexible, as long as it is sufficiently dimensionally-stable to be handled and introduced into a processing apparatus without substantially destroying its general structure or rendering the matrix unusable for its intended purpose in the sample manipulation procedure.

Non-limiting examples of suitable sufficiently dimensionally-stable chemical components include pellets, tablets or microtablets including a reagent, beads or microbeads for capture of proteins, magnetic beads, fluorescent beads, buffers or another chemical, a film chip that substantially dissolves in fluid, a dissolving beads, other thin films, a support film coated with a reagent layer, a lyophilized reagent or another lyophilized chemical, and so forth. As described in U.S. Provisional Patent Application No. 60/985,933 (Attorney docket No. 63696US002), which is incorporated herein by reference in its entirety, a reagent tablet or microtablet may be formed by compressing a reagent and matrix material. Other types of compressed and non-compressed chemical components are contemplated. The chemical components may have any suitable shape and size that permits the chemical component to be introduced into a processing device.

In addition, in accordance with an embodiment of the methods and systems described herein, a plurality of the chemical components may be manufactured and stored in a carrier. The carrier enables relatively easy transportation and storage of the chemical components, which, in some embodiments, may be relatively small in size (e.g., microtablets having a greatest dimension in a range of about 0.5 millimeters (mm) to about 5 mm). In some embodiments, chemical components including different reagents may be packaged in different carriers. The substantially dimensionally-stable form of the chemical components and storage in a carrier may also extend the shelf life of the reagents compared to a liquid form of the chemical components, which may be more difficult to transport and store.

Substantially dimensionally-stable chemical components that are preformed and incorporated into an automatic or semi-automatic placement technique may help increase the speed at which the processing device is assembled. Some previous techniques of incorporating chemistries into processing devices involve the dispensing of the chemistries in a liquid format into the processing device at the time the processing device is assembled. The liquid chemistries are substantially dried before completing assembly of the processing device. Depending on the amount of liquid deposited into the processing device, the drying process may be substantially time-consuming, and may take about 10 minutes to two hours or more. The drying time may increase if a large quantity of the chemistry is required, which may result in a larger amount of liquid that is introduced into the processing device and subsequently dried. The drying time may also be compounded if more than one chemistry is deposited into each processing device.

On the other hand, an assembly technique that involves placing a substantially dimensionally-stable chemical component into a processing device merely requires the time required to place the chemical component into the processing device and seal the processing device. The assembly time does not significantly change for larger quantities of chemicals, which may, for example, result in a larger chemical component. As described in further detail below, in some embodiments, a relatively high speed automated process may be used to pick and place the chemical components within the processing device. Thus, the assembly techniques and systems described herein result in relatively efficient assembly of a processing device and the desired chemistries in the form of a substantially dimensionally-stable chemical component.

Lower cost assembly sites are possible by separating the chemical component manufacture from the assembly process because in some cases, manufacturing of the chemical components may require a more specialized process than the assembly process. For example, introducing a liquid or another form of a chemical component into a processing device via a process that requires precise and accurate measurement of the quantity of the chemical may be burdensome and require relatively expensive machinery. In addition, as described in further detail below, introducing a chemical into the processing device as a liquid and subsequently drying the chemical may require a relatively clean operating environment in order to minimize potential contaminants in the chemical. Thus, by preparing the substantially dimensionally-stable chemical component to include a particular quantity of the desired chemicals before introduction into the processing device, the need to precisely and accurately measure the dosage of the chemical component at the processing device assembly site is substantially eliminated. Preparation of the chemical component prior to introduction into the processing device rather than introducing a chemical into the processing device in a liquid form and subsequently drying the liquid may reduce exposure to potential contaminants. In some embodiments, the prepackaged chemical components may be manufactured at one site and transported to another site at which the chemical components are assembled into a processing device.

In the existing techniques for assembly processing devices that include depositing a wet chemistry into a processing device, the chambers of the processing device are left exposed to the operating environment as the wet chemistry dries. As a result, there is an increased potential for contamination of the chemistries and process chamber as compared to the techniques described herein in which a substantially dimensionally-stable chemical component including the desired chemistries is placed within the processing device and the processing device is sealed relatively quickly, e.g., on the order of seconds, rather than minutes. In addition, some fluid mixtures may not properly dehydrate, e.g., by evaporating in a non-controlled fashion, where moisture is drawout from the outer surface of the droplet which causes the solid materials to migrate outward. Such fluid mixtures may not resuspend to its original form upon the addition of water again, and, accordingly, the performance of the processing device including such a dehydrated fluid mixture may be compromised. Designing the chemistries into substantially stable, dry, pre-measured, pre-tested “tablets” or other chemical components that are designed to rapidly dissolve helps mitigate the problems caused by depositing wet chemistries into a processing device.

Due to the potential for contamination and other factors, it may be difficult to perform quality control on the wet chemistry that is subsequently dried. In contrast, by preparing and packaging the chemical components prior to assembly into a processing device, the batch of chemical components may be more easily tested for quality (e.g., amount of chemistry in each component and/or presence of contaminants in the chemical component) prior to assembly with the processing device. For example, one or more chemical components from a batch, which may be, for example, the chemical components of a single carrier, may be tested. If one or more of the tested chemical components are unsuitable for integration into a processing device, the entire batch may be unsuitable, the tested component may be discarded or at least one other chemical component from the batch may be tested for quality to confirm that the batch is unsuitable.

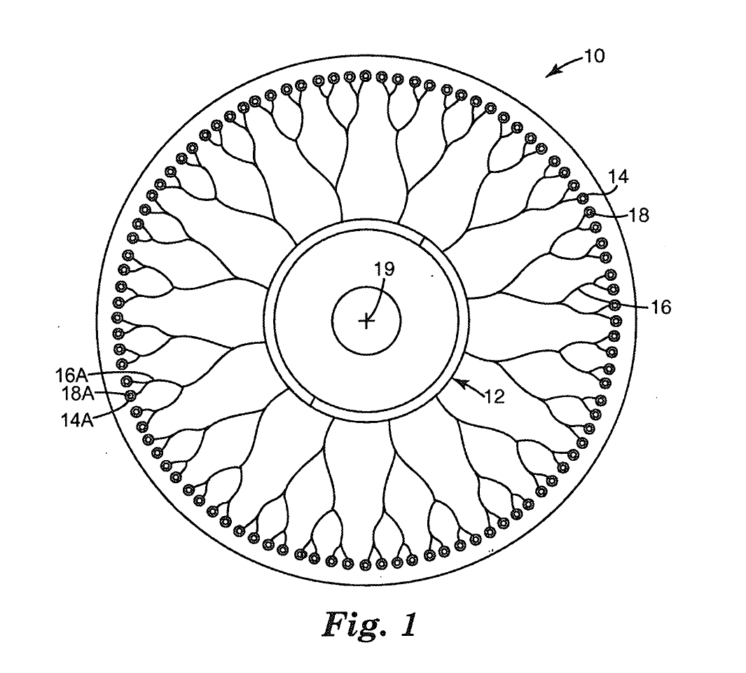

FIG. 1 is a schematic top view of processing device 10, which includes supply chamber 12, a plurality of process chambers 14, and a plurality of conduits 16 fluidically coupling supply chamber 12 with at least one process chamber 14. Process chambers 14 each define a volume for containing a fluid or a channel through which a fluid may pass through (e.g., capillaries, passageways, channels, grooves). A reagent microtablet 18 is disposed within each of process chambers 14. In the embodiment shown in FIG. 1, conduits 16 are each a microfluidic channel.

Processing device 10 is useful for processing an analyte, which may be in the form of a fluid (e.g., a solution, etc.) or a solid or semi-solid material carried in a fluid. For example, processing device 10 may include a chemical component useful for preparing an analyte for detection of a particular bacteria or other target microorganism of interest within the analyte. The analyte may be from a living (e.g., a human patient) or nonliving source (e.g., a food preparation surface). The analyte may be entrained in the fluid, in solution within the fluid, and so forth. Thus, reference to an “analyte” or “sample” refers to any fluid in which the analyte is or may be located, regardless of whether the analyte is, itself, a fluid or is contained within a carrier fluid (in solution, suspension, etc.). Furthermore, in some instances, analyte may be used to refer to fluids in which a target analyte (i.e., the analyte sought to be processed) is not present. For example, wash fluids (e.g., saline, etc.) may also be referred to as an analyte.

A user may introduce an analyte into supply chamber 12, which may then be introduced into at least one of process chambers 14 via the respective conduit 16. Any suitable technique may be employed to move the analyte from supply chamber 12 to the respective process chamber 14, such as via centrifugal forces generated by rotating processing device 10 about a center axis 20, gravitational forces (actual or induced), vacuum forces, thermal transfer techniques, as described in commonly-assigned U.S. Patent Application Ser. No. 60/871,611 (attorney docket number 62471US002) filed on Dec. 22, 2006 (Bedingham et al.), which is incorporated herein by reference in its entirety, or other suitable techniques. Although movement of fluids within processing device 10 is primarily described with reference to centrifugal forces generated by rotation of device 10, in other embodiments, any one or combination of techniques may be used to move fluid within processing device 10, e.g., a combination of rotational and gravitational forces.

After moving into one or more process chambers 14, the analyte may be processed to obtain a desired reaction, such as, but not limited to a polymerase chain reaction (PCR), ligase chain reaction (LCR), sustaining sequence replication, enzyme kinetic studies, homogeneous ligand binding assays, and other chemical, biochemical, or other reactions. A “chamber” as used herein should not be construed as limiting the chamber to one in which a process (e.g., PCR, Sanger sequencing, etc.) is performed. Rather, a chamber may include, e.g., a volume in which materials are loaded for subsequent delivery to another chamber as the processing device if rotated, a chamber in which the product of a process is collected, a chamber in which materials are filtered, and so forth.

In the embodiment shown in FIG. 1, upon introduction into at least one of the chambers 14, the analyte reacts with a reagent that is within microtablets 18. Process chamber 14A, conduit 16A, and reagent microtablet 18A are primarily referred to throughout the description of FIGS. 1-6. However, the description of process chamber 14A, conduit 16A, and reagent microtablet 18A are also applicable to each of the plurality of process chambers 14 and respective conduits 16 and reagent microtablets 18.

Microtablet 18A includes at least one type of reagent and a matrix material that may or may not be soluble. Fluid from the analyte or a fluid otherwise introduced into process chamber 14A may be used to at least partially dissolve microtablet 18A and release the reagent therein. In order to increase the speed of the dissolution of microtablet 18A, processing device 10 may be manipulated to encourage fluid flow around microtablet 18A. For example, processing device 10 may be rotated about center axis 20 in a particular pattern (e.g., accelerating or decelerating in a particular pattern). As another example, vacuum forces may be introduced into chambers 14 via supply chamber 12 or another source, and the release and application of the vacuum force may encourage the movement of fluid within process chamber 14A or the use of air compression chambers (not shown in FIG. 1) coupled to each of process chambers 14 may be used to move fluid back and forth as the air chambers undergo alternating centrifugal force that compresses the trapped air.

While processing device 10 is shown in FIG. 1 to have a circular disc shape, in other embodiments, processing device 10 may define any other suitable shape. In some embodiments, a shape of processing device 10 is selected to aid rotation of device 10. In addition, processing device 10 may include any suitable number of process chambers 14 and supply chambers 12. For example, while 96 process chambers 14 are shown in FIG. 1, in other embodiments, a processing device may include as few as one process chamber or more than 96 process chambers. Furthermore, in other embodiments, a process chamber may include multiple supply chambers, e.g., as shown and described below with respect to FIG. 8.

In some embodiments, processing device 10 may be a thermal transfer structure. The thermal transfer processing device 10 may be useful for reactions that require relatively precise thermal control (e.g., an isothermal process sensitive to temperature variations) and/or rapid thermal variations. Accordingly, in some embodiments, at least one of surface of processing device 10 defines a surface that is complementary to a base plate or thermal structure apparatus as described in, e.g., U.S. Pat. No. 6,734,401 titled ENHANCED SAMPLE PROCESSING DEVICES SYSTEMS AND METHODS (Bedingham et al.); U.S. Patent Application Publication No. 2007/0009391, titled COMPLIANT MICROFLUIDIC SAMPLE PROCESSING DISKS, filed on Jul. 5, 2005; and U.S. Patent Application Publication No. 2007/0010007, titled SAMPLE PROCESSING DEVICE COMPRESSION SYSTEMS AND METHODS, filed on Jul. 5, 2005. For example, in some embodiments, at least one of the major surfaces of processing device 10 may define a substantially flat surface.

In the illustrated processing device 10 of FIG. 1, supply chamber 12 is a single chamber. In other embodiments, supply chamber 12 may be divided into two or more subchambers that are isolated from each other. This allows a different material, for example a sample material or a buffer, to be introduced into each subchamber for distribution to the process chambers 14 by way of channels 16.

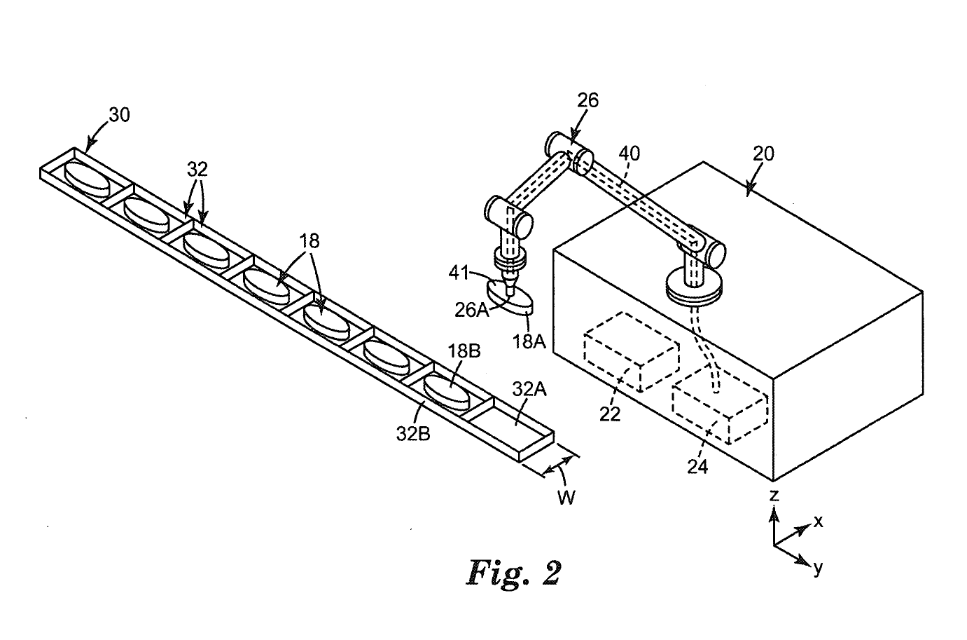

FIG. 2 is a schematic diagram illustrating an embodiment of a system for transferring tablets 18 into respective process chambers 14 of processing device 10 with the aid of placement device 20. In the embodiment shown in FIG. 1, placement device 20 includes controller 22, vacuum source 24, and robotic arm 26. Placement device 20 may be any device configured to “pick and place” tablets 18 or other chemical components within processing device 10. In the embodiment shown in FIG. 2, placement device 20 is an apparatus that includes a controllable arm 26 to retrieve a tablet 18 from carrier 30 and place the retrieved tablet 18 into processing device 10.

Although not shown in FIG. 2, in some embodiments, at least a portion of placement device 20 may be enclosed. For example, robotic arm 26, processing device 10, and at least the portion of carrier 30 from which a tablet 18 is removed may be enclosed within a housing, such as a plastic or glass housing. Controller 22 or another computing device may control the environment within enclosed space in order to control the environment in which tablets 18 are assembled with processing device 10. For example, controller 22 may maintain the humidity of the operating environment within a predetermined range in order to help preserve the integrity of tablets 18, which may be sensitive to relatively high levels of humidity (e.g., tablets 18 may include a hydrophilic material that absorbs water from the air, thereby changing the consistency of tablets 18). In addition, the enclosed space may be relatively clean in order to help prevent contamination of processing device 10. For example, the air within the enclosed spaced may be filtered to remove certain particles.

Placement device 20 is a “pick and place” device that places relatively small chemical components, such as, but not limited to, chemical components having a size of about 0.5 mm to about 20 mm, within processing device 10. Placement device 20 is configured to automatically position tablet 18 within a respective process chamber 14 of processing device 10 with relative precision and accuracy, because processing device 10 may include multiple relatively small chambers. In contrast, manual placement, e.g., by a human operator, may be time consuming, less accurate, and less precise.

In one embodiment, placement device 20 is an apparatus that utilizes surface mount technology (SMT). While SMT devices are conventionally used to “pick and place” relatively small electrical devices (e.g., resistors) on a circuit board in the electronics industry, the SMT device may be modified to place relatively small chemical components, such as lyophilized pellets, tablets or microtablets, within processing device 10. An example of a suitable SMT device that may be used is the Mydata TP9 UFP Pick & Place System, available from MYDATA automation, Inc. of Rowley, Mass. The Mydata TP9 UFP Pick & Place System has a placement speed of about 6,000 cycles per hour (CPH). In one example, the Mydata TP9 UFP Pick & Place System device was used to place a plurality of electrical resistors, which were representative of tablets 18, into processing device 10. The electrical resistors were about 1.6 mm by 0.80 mm by about 0.45 mm, and weighed about 2 grams per 1000 resistors. The Mydata TP9 UFP Pick & Place System was able to place approximately 96 resisters in the 96 process chambers 14 of device 10 in approximately one minute. However, a faster placement speed or a slower placement speed is possible with the Mydata TP9 UFP Pick & Place System.

An SMT device may be modified to place chemical components within a processing device. Modifications to an SMT device include, for example, incorporating a plurality of processing devices 10 and a plurality of tablets 18 or other chemical components rather than a plurality of circuit boards and electronic devices. A plurality of processing devices 10 may be delivered to the SMT device in trays or one or more carriers similar to carrier 30, but sized to receive processing devices 10, which are larger than tablets 18. The SMT device may also include a plate or another structure to support processing device 10, and, if necessary, move processing device 10. A plate of a commercially-available SMT device may be modified to support processing device 10 rather than, for example, a printed circuit board. Placement device 20 may align processing device 10 with robotic arm 26 via any suitable technique. In one embodiment, processing device 10 includes a fiducial marker, such as an indentation, protrusion, graphic marker, and so forth, which aligns with a particular location of placement device 20.

Processing device 10 may include fiducial markers, such as visual markers, grooves or protrusions, that allow robotic arm 26 to automatically identify processing device 10 and provide markers for aligning robotic arm 26 with processing device 10, e.g., to orient robotic arm 26 with device 10 or provide a starting point for assigning a coordinate system to processing device 10 (if necessary). The orientation of device 10 relative to robotic arm 26 may be useful for accurately and precisely orienting tablets 18 with process chambers 14, particularly when the geometry of tablets 18 are such that tablets 18 fit within process chamber 14 in a particular orientation. In addition, alignment of processing device 10 relative to robotic arm 26 may help placement device 20 verify that tablets 18 were properly placed within chambers 14. The fiducial markers may identify the type of consumable. Alternatively, if processing device 10 is carried on a tray or the like, the plate may be configured to automatically fit within a particular location relative to robotic arm 26.

As described in further detail below, tablets 18 or other chemical components may be delivered to an SMT device via carrier 30 that is wound around a reel. The reel may be mounted to the SMT device, as in current SMT devices that are used to pick and place electronic devices. If placement device 20 is configured to place different types of multiple chemical components and/or different processing devices are assembled via the SMT device, multiple reels may be mounted to the SMT device.

In a conventional SMT device that is used to fabricate electronic circuits, the device may apply solder paste to a printed circuit board prior to placing electronic devices on the circuit board. The use of soldering paste may be eliminated when the SMT device is used to pick and place tablets 18 within processing device 10. Other modifications are also contemplated.

Robotic arm 26 may be fixed and include at least one portion that is movable in the x-axis, y-axis, and/or z-axis directions. Alternatively, robotic arm 26 may be movable in the x-axis, y-axis, and/or z-axis directions.

Controller 22 of placement device 20 may include software executing on a processing device, hardware, firmware or combinations thereof. For example, controller 22 may include a computer, a microprocessor, a digital signal processor (DSP), an application specific integrated circuit (ASIC), a field programmable gate array (FPGA), discrete logic circuitry or the like. Controller 22 controls the movement of robotic arm 26 in at least one direction. In one embodiment, controller 22 controls robotic arm 26 along substantially x-axis, y-axis, and z-axis directions (orthogonal x-y-z axes are shown in FIG. 2) based on a coordinate system associated with a workspace. That is, controller 22 associates a workspace with a coordinate system, and when processing device 10 is placed within the workspace, the specific coordinates of process chambers 14 may be programmed (via software, firmware, hardware or combinations thereof) into controller 22. Controller 22 may then direct robotic arm 26 to process chambers 14 via the specific coordinates. In this way, robotic arm 26 may be a numerically controlled (NC) and/or a computer numerically controlled (CNC) robotic arm that minimizes, and, in some cases, eliminates operator intervention or control of robotic arm 26.

In other embodiments, controller 22 may utilize a system other than a coordinate system to control robotic arm 26. For example, processing device 10 may be mapped, e.g., as a graphic map, and controller 22 may rely on graphics to locate process chambers 14. As another example, each process chamber 14 of processing device 10 may include one or more fiducial markers, e.g., indentations, protrusions, graphic markers, and so forth, and controller 22 may align tip 26A of robotic arm 26 with process chamber 14A with the aid of the one or more fiducial markers.

Placement device 20 may include a user interface, such as a display and an input mechanism (e.g., an alphanumeric keyboard, peripheral pointing device, a limited set of buttons, etc.) or a touch screen display that enables an operator to interact with placement device 20. In the embodiment in which controller 22 controls the movement of robotic arm 26 based on a coordinate system, the operator may interact with the user interface to provide the coordinates for process chambers 14. For example, the operator may program the location of process chambers 14 with the aid of a video interface, whereby a video representation of processing device 10 is automatically associated with a coordinate system. The operator may select particular regions of processing device 10 via the user interface to indicate where each tablet 18 should be placed. Alternatively, the coordinates for process chambers 14 of one or more processing devices 10 may be stored within a memory of placement device 20 or another computing device coupled to placement device 20. An operator may then select the type of processing device from a list of stored processing devices, and controller 22 may automatically retrieve or receive the relevant coordinates for process chamber 14.

If more than one tablet 18 is placed within chamber 14A, controller 22 may direct robotic arm 26 to different regions of chamber 14A via coordinates or another system. In this way, different tablets 18 or chemical components may be placed at different regions within chamber 14A. For example, it may be desirable to place a certain tablet 18 closer to channel 16A or to stack chemical components in the z-axis direction.

In some embodiments, controller 22 controls robotic arm 26 to place tablet 18A directly on a surface (e.g., a bottom surface) of process chamber 14A. However, direct placement on a surface of process chamber 14A may require programming controller 22 for a particular processing device 10 and tablet 18A size. In other embodiments, robotic arm 26 may “drop” tablet 18A into chamber 14A, rather than placing tablet 18A directly on a particular surface of chamber 14A. That is, in another embodiment, controller 22 controls vacuum source 24 to release the vacuum pressure when tip 26A is substantially near, but not contacting chamber 14A. In this way, controller 22 may automatically compensate for the z-axis location of chamber 14A relative to tip 26A of robotic arm 26.

Placement device 20 is configured to place tablets 18 of many different sizes, weights, and configurations within processing device 10, as well as other types of processing devices. For example, while curvilinear tablets 18 are shown in FIG. 2, in other embodiments, placement device 20 may place chemical components having irregular shapes, straight edges (e.g., cubes), or spherical, cylindrical, triangular or pyramid-shaped chemical components. In some embodiments, tablets 18 may include a visible or otherwise detectable identifier, e.g., different shapes, markings (e.g., protrusions, graphic markings, fiducial markers, etc.), and/or colors that is specific to the type of chemistry within tablets 18. In this way, the visible or otherwise detectable identifiers may be easily identified by a robotic vision system, tactile system or other identifier locator of placement device or a manual assembler. In some embodiments, robotic arm 26 is configured to pick and place chemical components having a smallest dimension of about 0.10 mm to a greatest dimension of about 6 mm or greater, typically about 10 mm. Example weights of chemical components that robotic arm 26 is configured to handle include, but are not limited to, 0.4 grams per 1000 chemical components to about 45 grams per 1000 chemical components.

In addition, robotic arm 26 is configured to handle chemical components that are not compressed, such as a lyophilized pellet of a reagent, which may be more delicate than a compressed tablet 18. The vacuum force with which robotic arm 26 couples to the chemical component may be modified, depending on the type of chemical component. Thus, vacuum source 24 may exert a lower vacuum force with a lyophilized pellet or other non-compressed chemical components than with a compressed chemical component. In other embodiments, robotic arm 26 may couple to tablet 18A via a mechanical mechanism, e.g., arms or a basket that engage tablet 18A.

Tablets 18 are typically relative small, and, in some cases, may be microtablets, which may have a greatest dimension of about less than 5 millimeters (mm). In order to help handle tablets 18, tablets 18 may be grouped together and packaged in carrier 30. Carrier 30 may be useful for storing tablets 18, e.g., for transportation between a tablet manufacturing locating and a location in which tablets 18 are assembled with processing device 10. If desired, carrier 30 including tablets 18 may be stored within a storage unit (e.g., a sealed bag, box, jar, and so forth) that includes a desiccant in order to help minimize humidity and preserve tablets 18, which may include a hydrophilic material that is susceptible to absorbing water. In some cases, a disposable humidity history indicator may also be included in the package to provide an indication to a user of the humidity tablets 18 were exposed to during storage. Carrier 30 may be air permeable (e.g. may include a hole in pockets 32 or a permeable cover film) to permit desiccant to extract moisture. In other embodiments, carrier 30 may be hermetically sealed or disposed within a hermetic carrier system (e.g. foil or metalized plastic tray/tapes and cover films) that can protect tablets 29 from excessive humidity until tablets 18 are placed within processing device 10. Carrier 30 may also be useful for positioning tablets 18 relative to robotic arm 26, as described in further detail below.

As shown in FIG. 2, carrier 30 defines a plurality of pockets 32. Carrier 30 may formed of any suitable material, such as a plastic, paper (e.g., cardboard), foil or combinations thereof. Width W of carrier 30 may be selected to accommodate the size of tablets 18. In the case of microtablets 18, for example, width W may be between about 0.5 millimeters (mm) to about 8 mm. In the case of tablets or other relatively larger chemical components, width W may be 8 mm or greater, such as about 8 mm to about 200 mm.

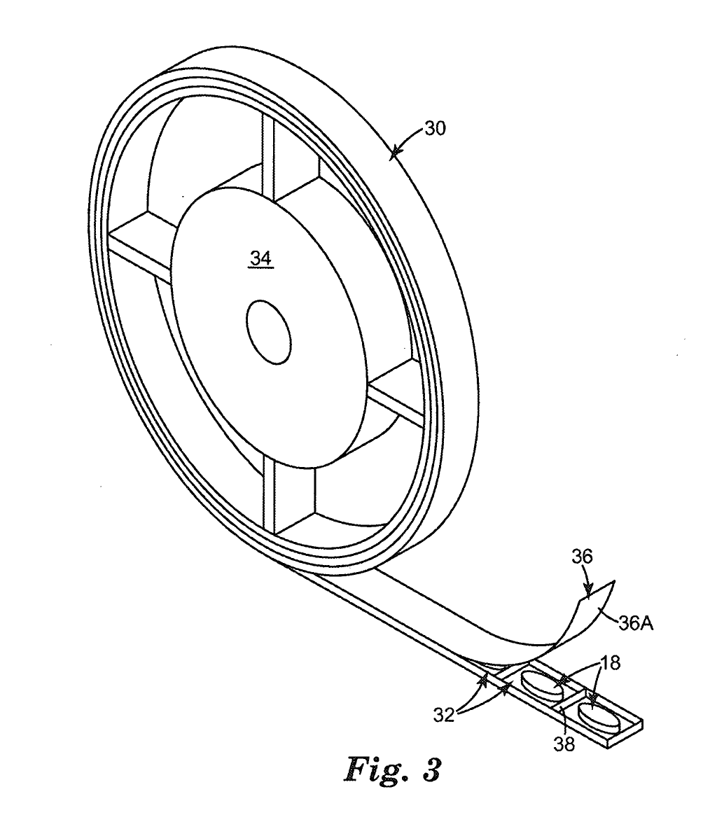

Width W of carrier 30 may also be based on the suitable size for integration into placement device 20. For example, as described with respect to FIG. 3, carrier 30 may be mounted about a reel and placement device 20 may be configured to receive the reel and feed carrier 30 through a device that removes a cover from carrier 30 in order to retrieve tablets 18 from pockets 32. A length of carrier 30, measured in a generally y-axis direction in FIG. 2 and substantially transverse to width W, may be any suitable length, and may be based on, for example, the number of tablets 18 packaged by carrier 30. In addition, a thickness of carrier 30 and depth of pockets 32, measured in a generally z-axis direction, may also be selected based on the size of tablets 18 and the requirements of placement device 20.

Pockets 32 define a space for receiving at least one tablet 18. As shown in FIG. 2, at least one tablet 18 is disposed within each pocket 32. For example, pocket 32A corresponds to tablet 18A, and tablet 18B is disposed in pocket 32B. In other embodiments, more than one tablet or other chemical component may be placed within a single pocket 32 at the same time as tablets 18 or after tablets 18 are placed within chamber 14A. As previously described, placement device 20 may be configured to retrieve chemical components from more than one carrier 30. In the embodiment shown in FIG. 2, pockets 32 are separated from each other via a wall or another dividing structure such that each pocket 32 defines a discrete space for tablets 18. In addition, the discrete space for tablets 18 defined by pockets 32 allow for indexed automation of robotic arm 26. That is, pockets 32 define a space with which controller 22 may align robotic arm 26 to locate each tablet 18 within carrier 30. Controller 22 may control the automatic indexing of carrier 30. For example, carrier 30 may be mounted on a reel that includes sprockets, which permits controller 22 to advance carrier 30 in known, discrete increments, as well as remove any cover film from carrier 30 in known amounts in order to expose each pocket 32.

In some embodiments, the interior surface of pockets 32 (i.e., the surfaces that tablets 18 may contact) may be substantially smooth in order to help prevent any abrasion to tablets 18. One surface of pocket 32 may be exposed such that tablet 18 may be removed therefrom. In this way, pockets 32 define exposed recesses. In order to help contain tablets 18 within the respective pockets 32, e.g., during transport of carrier 30, pockets 32 may each be sealed, e.g., via a tape, foil, thin film or other suitable material that does not react substantially with tablets 18. The tape, foil, thin film or other seal may extend across one pocket 32 or more than one pocket 32.

Compared to a process in which chemicals are deposited within process chamber 14A in a liquid form and subsequently dehydrated, the assembly process for processing device 14A including one or more chemical components is simplified and the assembly time is decreased when the chemical components are automatically placed within process chamber 14A. For example, if multiple tablets 18 are placed within each process chamber 14 of device 10, intermixing of the chemistries of two or more tablets within a single chamber 14 is minimized because substantially solid chemical components are placed within chambers 14. In contrast, depositing two or more liquids within chamber 14A may be result in more intermixing of the chemicals.

The interchangeability of carriers within an SMT device or another placement device 20 enables a single SMT device or placement device 20 to be used to assemble multiple types of processing devices. In addition, placement device 20 may be configured to receive two or more carriers 30 at a time, thereby supporting the relatively easy assembly of a processing device including more than one type of chemical component. Placement device 20 to place multiple chemical components within a single device or assemble multiple types of chemical components with the same or different processing devices. If placement device 20 is configured to place different types of multiple chemical components and/or different processing devices are assembled via placement device 20, the use of carriers that package substantially dimensionally-stable chemical components may permit relatively easy switching between chemical components. For example, one carrier 30 of chemical components may be changed out for another carrier 30 including another type of chemical component in the time that it takes to remove and replace carrier 30. In this way, placement device 20 and carrier 30 help reduce set-up time and changeover time for configuring placement device 20 to assemble different processing devices or assemble different types of chemical components with processing device 10.

In addition, because the chemical components are substantially dimensionally-stable, any possible contamination between different types of chemical components, e.g., via particles on robotic arm 26 is minimized. In some embodiments, the SMT device or another placement device 20 may be configured to receive multiple carriers 30 of chemical components, thereby supporting the relatively easy assembly of a processing device including more than one type of chemical component.

In one embodiment, as shown in FIG. 3, carrier 30 may be rolled onto reel 34 that may be integrated into a placement device 20. During a process in which processing device 10 and tablets 18 are assembled, controller 22 or another controller of placement device 20 may automatically advance reel 34 as each tablet 18 is removed from carrier 30. For example, reel 34 may define grooves, protrusions or other indicators of the relative position of reel 34, and controller 22 may control a device (e.g., an actuator motor) to rotate reel 34 as needed to advance carrier 30 and expose new tablets 18 as tablets 18 are removed from carrier 30. After tablets 18 are removed from carrier 30, carrier 30 may be wound around another reel, e.g., opposing reel 34, thereby assisting the advancement of carrier 30. Because each pocket 32 of carrier 30 defines a discrete space for one or more tablets 18, controller 22 may precisely and accurately rotate reel 34 to advance carrier 30 and expose each tablet 18. In some embodiments, walls 38 between pockets 32 may be substantially vertical (i.e., substantially along the x-axis direction in FIG. 2) or may be angled, e.g., to guide robotic arm 26 into the pockets 32.

As shown in FIG. 3, a cover 36, which may be a tape, foil, thin film or other suitable cover, may be removed from carrier 30 in order to expose tablets 18 and remove tablets 18 from carrier 30. Cover 36 may be applied to carrier 30 via any suitable technique, such as an adhesive, melt bonding, combinations of melt bonding and an adhesive, ultrasonic bonding, and so forth. In one embodiment, surface 36A of cover 36 includes a pressure sensitive adhesive that adheres to corresponding surfaces of carrier 30. In order to help prevent tablets 18 from adhering to surface 36A, however, it may be desirable to apply adhesive to carrier 30, such that the portion of tape 36A substantially aligning with the opening in pockets 32 does not have adhesive.

Furthermore, the pressure sensitive adhesive may be a single pressure sensitive adhesive or a combination or blend of two or more pressure sensitive adhesives. The pressure sensitive adhesive may be applied via a solvent coating, screen printing, roller printing, melt extrusion coating, melt spraying, stripe coating, or laminating processes, for example. The pressure sensitive adhesive may be provided in the form of a layer of pressure sensitive adhesive that may be provided as a continuous, unbroken layer between cover 30 and the opposing surfaces of carrier 30. Examples of some potentially suitable attachment techniques, adhesives, etc. may be described in, e.g., U.S. Pat. No. 6,734,401 entitled, “ENHANCED SAMPLE PROCESSING DEVICES SYSTEMS AND METHODS” (Bedingham et al.) and U.S. Pat. No. 7,023,168, entitled “SAMPLE PROCESSING DEVICES” (Bedingham et al.), which is incorporated by reference herein in its entirety. In embodiments in which cover 36 is melt bonded to carrier 30, cover 36 and the surface of carrier 30 to which it is attached may include, e.g., polypropylene or some other melt bondable material, to facilitate melt bonding.

One aspect of the invention relates to packaging chemical components within carrier 30. In one embodiment of packaging chemical components, an automated device including, for example, a computer-controlled robotic arm, may place the chemical components within pockets 32 of carrier 30 after the chemical components are formed. The same robotic arm or another computer-controlled apparatus may apply a cover 36 (FIG. 3) to substantially seal pockets 32 and protect the chemical components from contamination. In one embodiment, pockets 32 are hermetically sealed.

Returning now to FIG. 2, under the control of controller 22, robotic arm 26 may pick up tablet 18A from carrier 30, e.g., a vacuum force or a mechanical device (e.g., arms that grasp tablet 18A). In the embodiment shown in FIG. 2, robotic arm 26 includes a vacuum channel 40 that is coupled to vacuum source 24. Vacuum channel 40 extends to tip 26A or substantially near tip 26A of robotic arm 26. Controller 22 may control vacuum channel 40 to apply a vacuum force at tip 26A. The vacuum force at tip 26A creates a suction force that removes tablet 18A from carrier 30 and couples tablet 18A to robotic arm 26. Controller 22 may confirm that tablet 18A is coupled to robotic arm 26 by determining a change in pressure in vacuum channel 38.

A vacuum force may provide advantages over a mechanical device. For example, with a vacuum force, robotic arm 26A does not need to precisely and accurately align with a pocket 32A in order to retrieve the respective tablet 18A therefrom. Rather, the suction force from the vacuum force may be sufficient to pick-up tablet 18A as long as arm 26 is near tablet 18A.

In order to help prevent tablet 18A from being suctioned into vacuum channel 40, tip 26A may be sized and configured to be smaller than at least one major surface 41 of tablet 18A. In some embodiments, the major surface 41 of tablet 18A may be positioned within pocket 32 of carrier 30 such that tip 26A of robotic arm 26 first contacts major surface 41. Placement system 20 may be configured to receive different robotic arms 26 including different sized vacuum channels 40 in order to accommodate different sized chemical components.

In other embodiments, tablet 18A may be held at tip 26A of robotic arm 26 via an electrostatic charge, mechanical mechanism (e.g., movable arms) or a pressure sensitive adhesive. In the case of electrostatic charge, tablet 18A may be released from robotic arm 26 by reducing the electrostatic charge, attraction to an electrostatic charge within chamber 14A or by applying a positive gas pressure through a channel within robotic arm 26. In the case of a pressure sensitive adhesive, tablet 18A may be released from robotic arm 26 by contacting tablet 18A with a pressure sensitive adhesive within chamber 14A, contacting a pressure sensitive adhesive on tablet 18A to a surface within chamber 14A or by applying a positive gas pressure through a channel within robotic arm 26.

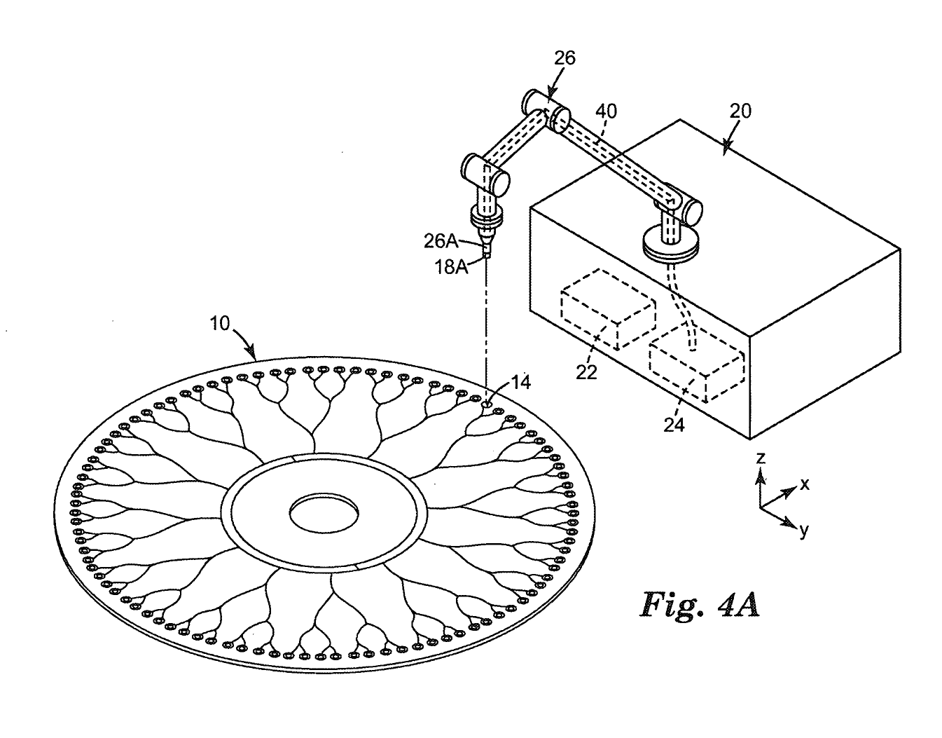

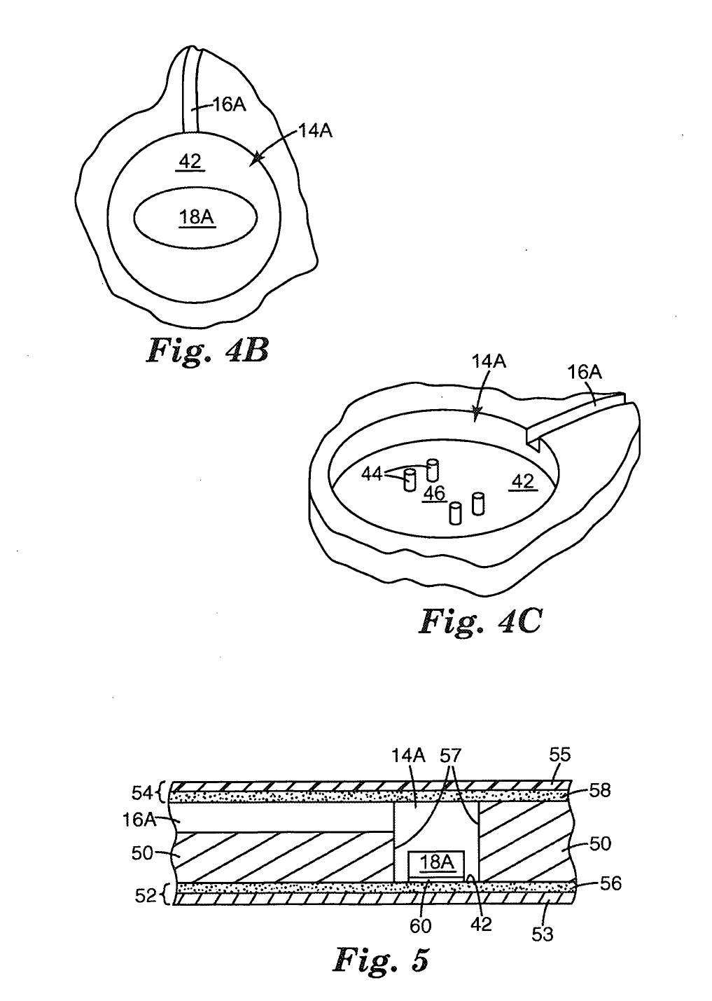

Under the control of controller 22, robotic arm 26 may move from a first position in which robotic arm 26 picks up tablet 18A (e.g., shown in FIG. 2) to a second position in which robotic arm 26 aligns with tablet 18A with process chamber 14A of processing device 10 (e.g., shown in FIG. 4). FIG. 4 illustrates tablet 18A aligned with process chamber 14A of device 10. In order to release tablet 18A, controller 22 controls vacuum source 24 to remove or minimize the vacuum force, such that tablet 18A is no longer coupled to tip 26A of robotic arm 26.

FIG. 4B is a schematic illustration of chamber 14A and tablet 18A. As shown in FIG. 4B and described in co-pending Provisional Patent Application No. 60/985,933 (Attorney Docket No. 63696US002), filed on the same date as the present disclosure, tablet 18A is sized to fit within chamber 14A. Accordingly, after vacuum source 24 releases the vacuum force or minimizes the vacuum force to decouple tablet 18A from tip 26A of robotic arm 26, tablet 18A is placed within chamber 14A.

As described in co-pending Provisional Patent Application No. 60/985,933 (Attorney Docket No. 63696US002), in some embodiments, tablet 18A may include a lubricant to aid in tabletting, in which case, tablet 18A may be relatively slippery and may not be inclined to stay in place within process chamber 14A, particularly if tablet 18A includes a curved surface that promotes movement. In some embodiments, in order to help prevent tablet 18A from being displaced from process chamber 14A, a tablet receiving surface 42 of each process chamber 14 may include an adhesive that contacts tablet 18A. Examples of adhesives are described in further detail with respect to FIG. 5.

FIG. 4C is a schematic illustration of another embodiment of process chamber 14A, which may be representative of other process chambers 14 of processing device 10. In the embodiment shown in FIG. 4C, process chamber 14A includes holding members 44 that engage with tablet 18A and substantially hold tablet 18A within process chamber 14A. Holding members 44 may be, for example, prongs or other structures that extend from bottom surface 42 of chamber 14A. Controller 22 of placement device 20 may be programmed to control robotic arm 26 to place tablet 18A within the inner space 46 defined by holding members 44. Holding members 44 are spaced from each other in order to increase the surface area of tablet 18A that is exposed to fluids during operation of processing device 10. Thus, in some cases, it may be desirable to decrease the size of holding members 44 and increase the surface area of tablet 18A that is exposed to fluids in order to increase the dissolution rate of tablet 18A during operation of processing device 10.

FIG. 5 is a partial cross-sectional view of processing device 10 and illustrates process chamber 14A, channel 16A, and tablet 18A. In the embodiment shown in FIG. 5, processing device 10 is comprised of multiple layers, including a substrate 50, a first layer 52, and a second layer 54. Substrate 50, first layer 52, and second layer 54 are preferably bonded or attached together to contain a fluid (e.g., an aqueous fluid) without leakage of the fluid through the bond or attachment between substrate 50 and first layer 52 or second layer 54. The bond or attachment may be, for example, a pressure sensitive adhesive, ultrasonic welding, hot melt adhesive, thermoset adhesive, a thermal bond or static charge. The type of bond or attachment may be selected based on the anticipated conditions for using tablet 18A. For example, a pressure sensitive adhesive may be selected if tablet 18A is to be used in an aqueous environment. In the embodiment shown in FIG. 5, optional bonding layer 56 may bond first layer 52 to substrate 50, and optional bonding layer 58 may bond second layer 54 to substrate 50.

Chamber 14A of device 10 is in fluid communication with channel 16A, which is also in fluid communication with supply chamber 12 (FIG. 1). As previously described, supply chamber 12 may supply a fluid (e.g., a sample material, a buffer, or the like) to channels 16 and chambers 14 of device 10. In the embodiment shown in FIG. 5, channel 16A is formed in substrate 50 and enclosed by second layer 54. In other embodiments, channel 16A may be on an opposite side of substrate 20 enclosed by first layer 52.

First layer 52 includes support layer 53 and second layer 54 includes support layer 55. Support layers 53 and 55 can each be comprised of one layer or multiple layers, can be a polymeric film such as described herein for the support film, can be a metallic layer, or a combination of a polymeric film and a metallic layer. Support layers 53 and 55 may or may not be the same. When support layers 53 and/or 55 are metallic, the respective optional bonding layers 56, 58 may be present to separate process chamber 14A from the metal of the metallic layer. In embodiments in which detection is made via fluorescence detection or color change detection within process chamber 14A, it may be desirable for at least one of support layers 53 and 55 to be formed from a nonmetallic layer in order to provide the capability of detecting fluorescence through the respective layer 53 and 55.

In FIG. 5, tablet 18A has been placed within process chamber 14A such that tablet contacts first layer 52. In the embodiment shown in FIG. 5, tablet 18A is adhered to bottom surface 42 of process chamber 14A by an optional pressure sensitive adhesive layer 60. Controller 22 of placement device 20 (FIG. 2) may control robotic arm 26 to position tablet 18A on optional adhesive layer 60, e.g., by placing tablet 18A in contact with adhesive layer 60 or by releasing tablet 18A over adhesive layer 60 such that tablet 18A “drops” onto adhesive layer 60. In another embodiment that may be used in addition to or instead of adhesive layer 60 placed on first layer 54 of processing device 10, tablet 18A may include an adhesive layer that contacts bottom surface 42 of process chamber 14A. For example, placement device 20 may include a robotic arm that places a pressure sensitive adhesive layer on tablet 18A prior to placing tablet 18A within process chamber 14A. In other embodiments, placement device 20 may place tablet 18A in process chamber 14A so as contact any one of the walls of the chamber 14A, including second layer 54 or sidewalls 57.

Instead of or in addition to optional adhesive layer 60, bonding layer 56 may be an adhesive layer that is configured to adhere tablet 18A to first layer 52. In yet another embodiment, optional bonding layer 58 may adhere tablet 18A to second layer 54 instead of or in addition to a separate adhesive layer 60. Optional bonding layers 56 and 58 and optional adhesive layer 60 may be any suitable bonding material, such as a pressure sensitive adhesive, hot melt adhesive, thermoset adhesive, other adhesives or other thermal bonds.

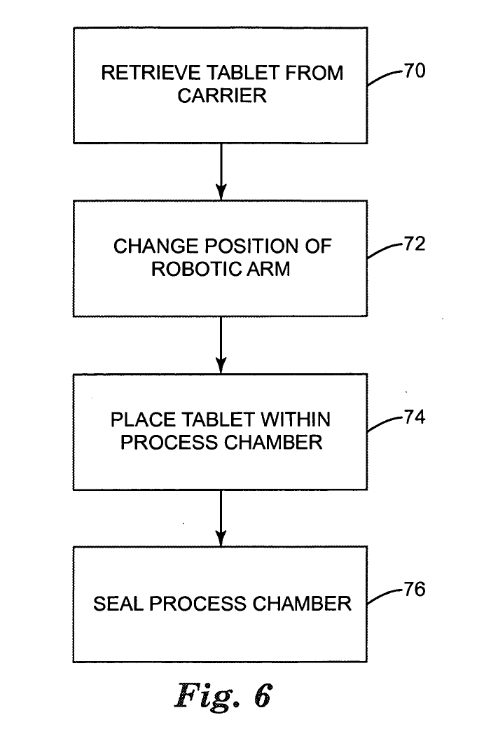

FIG. 6 is a flow chart illustrating an example technique for placing a chemical component, such as a tablet, within a processing device. As described above, the processing device may be processing device 10, which is a substantially self-contained device that includes one or more chemical components for sample preparation, detection or otherwise carrying out a useful reaction or combinations thereof. In some embodiments, each chemical component may include chemicals for a single reaction, while in other embodiments, each chemical component may include chemicals for multiple reactions. While the technique shown in FIG. 5 is described with respect to a tablet 18A in FIGS. 1-4C, in other embodiments, the technique may be applied to other chemical components.

In embodiments in which tablets 18 are stored within carrier 30, under the control of controller 22, robotic arm 26 retrieves tablet 18A from carrier 30 (70) prior to placing tablet 18A within processing device 10. Controller 22 may align robotic arm 26 with tablet 18 with the aid of pockets 32 of carrier 30, which each define a discrete space for indexing robotic arm 26 with carrier 30. Controller 22 then moves robotic arm 26 to substantially align tablet 18A with the respective process chamber 14A of processing device 10 (72). As previously described, in some embodiments, controller 22 locates process chamber 14A with the aid of coordinates.

Once robotic arm 26 is positioned substantially near process chamber 14A, controller 22 may control robotic arm 26 to release tablet 18A, thereby placing tablet 18A within the process chamber 14A (74). In some embodiments, placement device 20 or another device may at least partially seal process chamber 14A after tablet 18A is placed within process chamber 14A (76). At least partially sealing includes placing a cover film, sheet or other layer at least partially over an opening of chamber 14A while allowing a pathway for moving a fluid into chamber 14A. The pathway may include, for example, a channel connected to chamber 14A, or the pathway may be formed by piercing the cover film, sheet or layer to access chamber 14A. In some embodiments, process chamber 14A may be sealed to substantially contain fluids within chamber 14A. For example, placement device 20 may include a film (e.g., second layer 54 in FIG. 5) that is laminated to a top surface of processing device 10 after tablets 18 are placed within some or all of the process chambers 14. The film may be stored in placement device 20 in a roll form (with or without a backing).

Alternatively, processing device 10 include one or more tablets 18 within at least one of process chambers 14 may be automatically transferred to another workstation that at least partially seals one or more of the process chambers 14.

A substantially similar process may be repeated for each tablet 18. If multiple tablets 18 are introduced into processing device 10, placement device 20 or another device may seal multiple process chambers 14 after more than one tablet 18 is placed within processing device 10. In addition, if multiple tablets are disposed within each chamber 14 or two or more process chambers 14 of processing device 10 include different tablets, placement device 20 may be configured to “pick and place” more than one type of tablet. For example, if placement device 20 is a SMT device, multiple reels of carriers 30 may be mounted to the SMT device.

The techniques and systems for placing one or more chemical components within a processing device are described with respect to processing device 10 (FIG. 1), in other embodiments, the chemical component placement techniques and systems may be applied to other types of processing devices. For example, a chemical component may be placed in processing devices similar to those described in, e.g., U.S. Patent Application Publication Nos. 2005/0126312 (Bedingham et al.); 2005/0129583 (Bedingham et al.); 2007/0009391 (Bedingham et al.); as well as U.S. Pat. Nos. 6,627,159 (Bedingham et al.), 6,734,401 (Bedingham et al.), 6,987,253 B2 (Bedingham et al.), 6,814,935 (Harms et al.), 7,026,168 (Bedingham et al.), and 7,192,560 (Parthasarathy et al.), which are each incorporated herein by reference in their entireties. The documents identified above all disclose a variety of different constructions of processing devices that may include a chemical component. The devices may preferably include fluid features designed to process discrete microfluidic volumes of fluids, e.g., volumes of 1 milliliter or less, 100 microliters or less, or even 10 microliters or less.

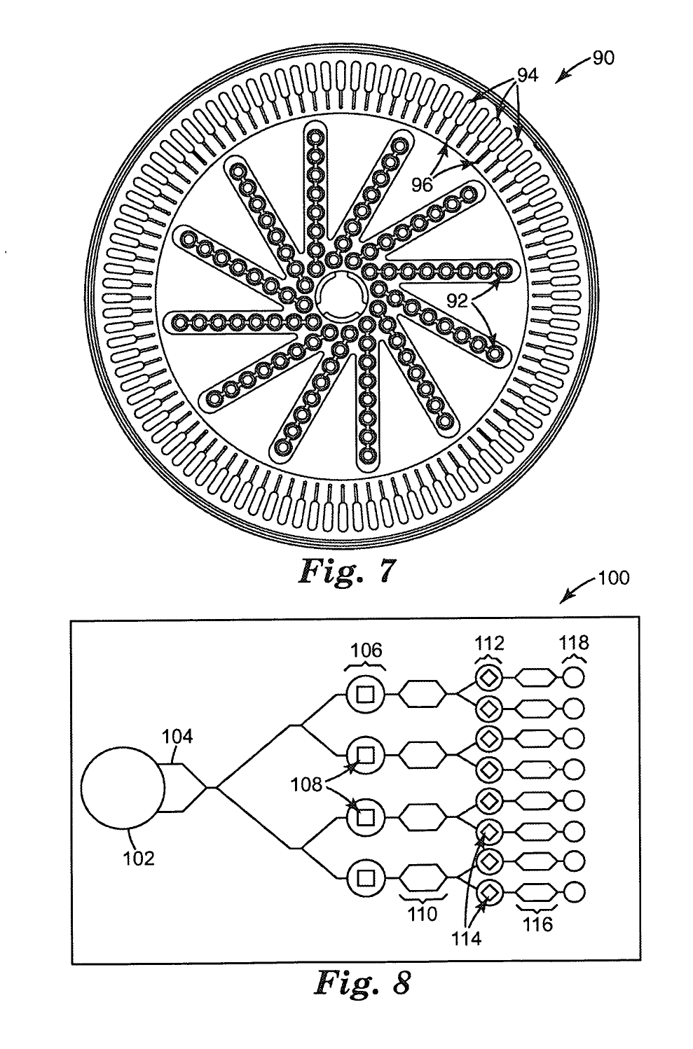

In addition, while processing device 10 including a single supply input chamber 12 is primarily described above, in other embodiments, a chemical component may be placed within a processing device including a plurality of supply input chambers in accordance with the systems and techniques described herein. FIG. 7 is a schematic diagram of an embodiment of a microfluidic processing device 90 that includes a plurality of input wells 92, a plurality of process chambers 94 coupled to a respective input well 92 via a microfluidic channel 96, which includes an inner channel, a via, and an outer channel (not shown in FIG. 7). Processing device 10 is described in further detail in commonly-assigned U.S. Patent Application Publication No. 2007/0009391, entitled, “COMPLIANT MICROFLUIDIC SAMPLE PROCESSING DISK” (Bedingham et al.), which is incorporated herein by reference in its entirety.

A chemical component may be placed within at least one of process chambers 94 using any of the techniques described above. For example, in one embodiment, placement device 20 (FIGS. 2 and 4A) may store coordinates for each of process chambers 94 of processing device 90 when processing device 90 is within a workspace of placement device 20. An operator may load one or more carriers 30 including one or more chemical components, e.g., tablets 18, and one or more microfluidic processing devices 90 into placement device 20. The operator may input the type of microfluidic processing device 90 that has been introduced into placement device 20, and controller 22 may access the coordinates for each of process chambers 94 from a memory of placement device 20. Alternatively, the operator may provide the relevant coordinates to controller 22. Because microfluidic processing device 90 is held in a known position relative to robotic arm 26 within the workspace of placement device 20, the coordinates provide sufficient direction for controller 22 to control robotic arm 26 during the placement of the chemical components within one or more of process chambers 94.

While both processing devices 10 (FIGS. 1-4) and 90 (FIG. 7) have a single “tier” of process chambers 14 such that fluid does not flow past each process chamber 14 or substantially all reactions take place within a single process chamber 14, in other embodiments, a chemical component may be placed within a processing device that includes two or more process chambers provided in a sequential relationship. The process chambers may be separated by a fluid control structure, such as a laser valve or another type of valve. FIG. 8 is a schematic illustration of processing device 100, which includes multiple process chambers in a sequential relationship. While one set of process chamber is shown in FIG. 8, in other embodiments, a plurality of sets of process chambers arranged similarly to that shown in FIG. 8 may be repeated about a common axis, as with processing device 10 and process chambers 14. An example of processing device 100 that includes fluid structures with multiple, connected process chambers is described in U.S. Pat. No. 6,734,401, entitled “ENHANCED SAMPLE PROCESSING DEVICES SYSTEMS AND METHODS,” (Bedingham et al.), which is incorporated herein by reference in its entirety.

As shown in FIG. 8, a sample loading chamber 102 is provided to receive, e.g., a starting sample material. The array and one illustrative method of using the array will be described below. The illustrative method involves PCR amplification, followed by Sanger sequencing to obtain a desired end product. This combination of processes is, however, intended to be illustrative only and should not be construed as limiting the types of processing devices in which a chemical component may be placed in accordance with the techniques and systems described herein.

In one example, a starting sample material, such as lysed blood cells, is provided in sample loading chamber 102. Filter 104 may be provided to filter the starting sample material as it moves from the loading chamber 102 to first tier of process chambers 106. Filter 104 is, however, optional and may not be required depending on the properties of the starting sample material. In one embodiment, first process chambers 106 includes chemical component 108, which includes a suitable PCR primers. Each of first process chambers 106 may include the chemical component 108 or different chemical components, depending on the nature of the investigation being performed on the starting sample material. One alternative to providing the primers in first process chambers 106 before loading the sample is to add a suitable primer to the loading chamber 102 with the starting sample material (provided that the primer is capable of passing through the filter 104, if present). In FIG. 8, as well as the other figures of the disclosure, the chemical components are not shown to scale relative to the process chambers.

After locating the starting sample material and any required primers in first process chambers 106 and dissolving chemical components 108, the materials in first process chambers 106 are thermally cycled under conditions suitable for PCR amplification of the selected genetic material. After completion of the PCR amplification process, the materials in each of first process chambers 106 may be moved through filter chamber 110 to remove unwanted materials from the amplified materials, e.g., PCR primers, unwanted materials in the starting sample that were not removed by filter 110, etc. In the embodiment shown in FIG. 8, each process chamber 106 is fluidically coupled to one filter chamber 110. The filter chambers 110 may, for example, contain size exclusion substances, such as permeation gels, beads, etc. (e.g., those available under the trade designations MicroSpin or Sephadex from Amersham Pharmacia Biotech AB, Uppsala, Sweden).

After clean-up of the sample materials in filter chambers 110, the filtered PCR amplification products from each of the first process chambers 106 are moved into a pair of multiplexed second process chambers 112 for, e.g., Sanger sequencing of the genetic materials amplified in the first process chambers 106 through appropriate control of the thermal conditions encountered in second process chambers 112. Disposed within each of second process chambers 112 is a chemical component 114, which may be used for Sanger sequencing.

Placement device 20 (FIGS. 2 and 4A) may place chemical component 114 within each of second process chambers 112 prior to, during or after chemical components 108 are placed within first process chambers 108. Chemical components 108 and 114 are different, and, accordingly, may be Packaged within different carriers that are coupled to placement device 20. If chemical components 108 and 114 are placed within device 100 at substantially the same time or both carriers for the chemical components 108 and 114 are coupled to placement device 20 at substantially the same time, controller 22 of placement device 20 may control robotic arm 26 to remove the desired chemical component 108 or 114 from the respective carriers. An operator may specify which chemical component 108 or 114 is to be placed within a process chamber 106 or 112, such as by placing the reels including the chemical component carriers in a particular order on placement device 20. Other techniques are also contemplated.

After the desired processing has been performed in second process chambers 112, the processed material (Sanger sequenced sample material if that is the process performed in second process chambers 112) is moved from each of second process chambers 112 through another set of filter chambers 116 to remove, e.g., dyes or other unwanted materials from the product of second process chambers 112. The filtered product is then moved from the filter chambers 116 into output chambers 118, where the product may be removed.

Chambers 102, 106, 112, and 118 may be arranged generally radially on device 100 such that rotation of device 100 will move materials from the loading chamber 102 towards the output chambers 118. For example, two or more of the process chamber arrays illustrated in FIG. 8 may be arranged on a single device, with the loading chambers 102 of each array located closest to the axis of rotation such that the materials can be moved through the array by centrifugal forces developed during rotation. Alternatively, the arrays may be located on a device that is held in a manner that allows rotation of device containing the array such that centrifugal forces move the materials from the loading chamber 102 towards the output chambers 118. Loading of sample materials into process chambers using centrifugal force is also described, for example, in U.S. Pat. No. 6,627,159, entitled, “CENTRIFUGAL FILLING OF SAMPLE PROCESSING DEVICES” (Bedingham et al.).

Various embodiments of the invention have been described. These and other embodiments are within the scope of the following claims. For example, although various constructions of illustrative embodiments of processing devices are described above, the chemical component placement techniques and systems may be used with other types of processing devices.

1. A method comprising:
introducing a substantially dimensionally-stable chemical component into a chamber of a sample processing device; and
at least partially sealing the chamber of the sample processing device.

2. (canceled)

3. The method of claim 1, wherein the chemical component comprises at least one of:
a powder in a substantially compact form, and
a quantity of a chemical for at least one of sample preparation, detection or analysis.

4. (canceled)

5. The method of claim 1, wherein introducing the chemical component into the chamber of the sample processing device comprises placing the chemical component into the chamber via surface mount technology.

6. The method of claim 1, wherein introducing the chemical component into the chamber of the sample processing device comprises controlling a robotic arm to place the chemical component into the chamber.

7. (canceled)

8. The method of claim 1, further comprising removing the chemical component from a carrier including a plurality of chemical components.

9. (canceled)

10. The method of claim 1, wherein introducing the chemical component into the chamber of the sample processing device comprises:
determining a type of the sample processing device; and
determining a location of the chamber based on the type of sample processing device.

11. The method of claim 1, wherein the chamber comprises an adhesive layer, and introducing the chemical component into the chamber comprises placing at least a portion of the chemical component on the adhesive layer.

12. (canceled)

13. The method of claim 1, wherein the chemical component comprises a first chemical component and the chamber comprises a first chamber, the method further comprising introducing a second chemical component into a second chamber of the sample processing device.

14. The method of claim 1, wherein at least partially sealing the chamber of the sample processing device comprises applying a layer of material at least partially over an opening of the chamber.

15. The method of claim 1, wherein the chemical component is at least partially soluble in water.

16. (canceled)

17. The method of claim 1, wherein the chemical component comprises at least one of a microtablet comprising a reagent or a support film coated with a reagent layer.

18. The method of claim 1, wherein the chemical component comprises at least one of a tablet, microtablet, lyophilized pellet, bead or a film.

19-20. (canceled)

21. A method comprising:
forming a plurality of substantially dimensionally-stable chemical components, the chemical components comprising at least one sample preparation or detection chemical for a sample processing device; and
packaging the plurality of chemical components in a carrier, wherein the carrier defines a plurality of pockets for receiving at least one chemical component.

22-23. (canceled)

24. An assembly comprising:
a carrier;
a plurality of chemical components disposed within the carrier;
a sample processing device;
a robotic arm; and
a controller to control the robotic arm to transfer at least one of the plurality of chemical components from the carrier to the sample processing device.

25. The assembly of claim 24, wherein the carrier defines a plurality of pockets, each of the plurality of pockets including at least one of the plurality of chemical components.

26. (canceled)

27. The assembly of claim 24, wherein the carrier is wound around a reel.

28. The assembly of claim 24, wherein the carrier comprises a first carrier and the plurality of chemical components comprises a first plurality of first chemical components, the assembly further comprising a second carrier and a second plurality of second chemical components different than the first chemical components, wherein the controller controls the robotic arm to transfer at least one of the second chemical components from the second carrier to the sample processing device.

29. (canceled)

30. The assembly of claim 24, the sample processing device defining a plurality of chambers, wherein the controller controls the robotic arm to transfer at least two of the plurality of chemical components from the carrier to respective chambers of the plurality of chambers, the assembly further comprising a memory to store a location of each of the plurality of chambers within a workspace associated with the robotic arm.

31. (canceled)

32. The assembly of claim 24, wherein the chemical component comprises an identifier that permits identification of the chemical component.

33. The assembly of claim 24, wherein the processing device comprises a fiducial marker, wherein the controller aligns the robotic arm with the processing device via the fiducial marker.
